(19) United States
(12) Patent Application Publication
(10) Pub. No.: US 2006/0205826 A1
(43) Pub. Date: Sep. 14, 2006

(54) Method of using carotenoids in the prevention of cognitive decline and for other neuroprotection functions

(76) Inventors: Francisco Javier Romero, Valencia (ES); Francisco Bosch-Morell, Alfara Del Patriarca (Valencia) (ES); Maria Miranda, Valencia (ES)

Correspondence Address: DAVIS, BROWN, KOEHN, SHORS & ROBERTS, P.C.;THE FINANCIAL CENTER, 666 WALNUT STREET, SUITE 2500, DES MOINES, IA, 50309-3993, US

(21) Appl. No.: 11/076,465

(22) Filed: Mar. 9, 2005

(51) Int. Cl. A61K 31/015

(52) U.S. Cl. 514/763

(57) ABSTRACT

Administration of carotenoids provide a prophylactic and/or therapeutic effect to subjects who are suffering from or at risk to develop cognitive decline or other neurological effects, such as diabetic complications, especially those related to neural tissues like retinopathy, peripheral neuropathy and central nervous system.

### BACKGROUND OF THE INVENTION

The invention relates generally to the use of carotenoids to provide neuroprotective effects and, more specifically, to the use of lutein and other carotenoids to treat or ameliorate cognitive decline and provide other neuroprotection functions.

Diabetes mellitus is an endocrine disorder of carbohydrate metabolism resulting primarily from inadequate insulin release (Type 1 insulin-dependent diabetes mellitus) or insulin insensitivity coupled with inadequate compensatory insulin release (Type 2 non-insulin-dependent diabetes mellitus). Though strict glycemic control is desirable to prevent diabetes complications, this is not always achievable. Thus, adjuvant therapies are needed to help in preventing or delaying the onset of diabetic complications.

It has been repeatedly suggested that oxidative stress may play an important role in the pathogenesis of late diabetes complications (Baynes 1996), though it is not clear yet whether increased oxidative stress has a primary role in the pathogenesis of diabetic complications, or it is simply the consequence of the presence of complications (Baynes 1999).

Hyperglycemia has the effect of reducing antioxidant levels and concomitantly increasing the production of free radicals, which contribute to the tissue damage associated with diabetes. This increase in free radicals is known to lead to an alteration in the redox potential of the cell and the subsequent activation of redox-sensitive genes.

Reactive oxygen species activate the transcription factor nuclear factor-kappaB (NF-kappaB), which in turn activates a variety of target genes linked to the development of diabetic complications. In retina it is demonstrated (Kowluru, 2003) that activation of retinal NF-KB in diabetes is an early event in the development of retinopathy, and it remains active when the retinal capillary cell death is accelerating, and histopathology is developing.

Diabetes mellitus has long been considered a risk factor for the development of vascular dementia. Epidemiologic evidence has suggested that diabetes mellitus significantly increases risk for the development of Alzheimer's disease, independent of vascular risk factors (Grossman, 2003). Diabetic patients have impaired learning/memory, brain atrophy, and two-fold increased risk of dementia. The cause of cognitive disturbances that progress to dementia is unknown (Lupien et al., 2003). One of the mechanisms by which hyperglycemia causes neural degeneration is via the increased oxidative stress that accompanies diabetes. Metabolic and oxidative insults often cause rapid changes in glial cells (Baydas et al., 2003). Astrocytes are critical for normal central nervous system function, and alterations in their activity could contribute to diabetes-related disturbances in the brain. Coleman et al. (2004) demonstrated that diabetes resulted in a significant decrease in GFAP protein levels in the hippocampus and cerebellum 4 weeks after the induction of diabetes in rats and in the cerebral cortex, hippocampus and cerebellum by 8 weeks. Other experimental studies (Li and Sima, 2004) have suggested that neuronal apoptosis may play an important role in neuronal loss and impaired cognitive function. In addition, in hippocampus of streptozotocin-treated rats, besides the strong increase in oxygen reactive species, there is also a persistent activation of NF-kappaB (Aragno et al, 2002).

Neurogenesis is primarily a developmental process that involves the proliferation, migration, and differentiation into neurons of primordial CNS stem cells (Gage, 2000). Neurogenesis declines until it ceases in the young adult mammalian brain with two exceptions: the olfactory bulb (OB) and the hippocampus produce new neurons throughout adult life. Reduction in the number of newly generated neurons in the dentate gyrus (DG) (through administration of an antimitotic agent) is associated with impairment in hippocampal-dependent tasks (Shors et al., 2001). A chronic, abnormal clearance process in the hippocampus may lead to memory disorders in the mammalian brain. Therefore, it seems that DG neurogenesis does play a role in cognitive functioning.

There is no standard drug therapy for cognitive decline. Several experimental agents may provide improvements in cognitive function. These agents are used specifically to facilitate learning and memory and might prevent the cognitive deficits associated with dementias. Such drugs currently being investigated include ergoloid mesylates (for example, the Novartis products Hydergine® and Gerimal) idebenone (a synthetic analogue of CoQ10 marketed as a “smart drug”), and derivatives of an inhibitory brain chemical called GABA (vinpocetine and bifemelane, which are also known by the investigational drug names piracetam, oxiracetam, and nebracetam).

In the elderly populations which eat a typical Mediterranean diet, high intake of monounsaturated fatty acids (e.g., olive oil) has been associated with protection against age-related cognitive decline in preliminary research (Soffrizi, et al., 1999). However, the monounsaturated fatty acid content of this diet might only be a marker for some other dietary or lifestyle component responsible for a low risk of age-related cognitive decline.

Caffeine may improve cognitive performance. Higher levels of coffee consumption were associated with improved cognitive performance in elderly British people in a preliminary study (Jarvis, 1993). Older people appeared to be more susceptible to the performance-improving effects of caffeine than were younger people. Similar but weaker associations were found for tea consumption. These associations have not yet been studied in clinical trials.

Animal studies suggest that diets high in antioxidant-rich foods, such as spinach and strawberries, may be beneficial in slowing age-related cognitive decline (Joseph, et al., 1998). Among people aged 65 and older, higher vitamin C and beta-carotene levels in the blood have been associated with better memory performance (Pering, et al., 1997).

### SUMMARY OF THE INVENTION

The invention consists of the administration of carotenoids to subjects who are suffering from or at risk to develop cognitive decline or other neurological effects, such as diabetic complications, especially those related to neural tissues like retinopathy, peripheral neuropathy and central nervous system.

### BRIEF DESCRIPTION OF THE DRAWINGS

FIG. 1 includes a chart of body weight changes (FIG. 1a), blood levels of glucose (FIG. 1b), glycated haemoglobin (FIG. 1c) and malondialdehyde concentration in blood from control, control+lutein, diabetic, diabetic+lutein and diabetic+insulin mice.

FIG. 2. includes charts of GPx activity (a), GSH (b) and MDA (c) concentrations, and b-wave amplitude (d) of the electroretinograms, in eyes from control, control+lutein, diabetic, diabetic+lutein and diabetic+insulin mice.

FIG. 3 includes a chart of GPx activity (a), GSH (b) and MDA (c) concentrations in hippocampus, and hippocampal-dependent task latencies (d, cf. Material and Methods), in control, control+lutein, diabetic, diabetic+lutein and diabetic+insulin mice.

FIGS. 4a and 4b are charts which show the effects of lutein on NF-kB activity in retina and hippocampus mice 15 days post alloxan treatment.

FIG. 5 is a chart showing the effects of lutein on survival rates of rats having diabetes.

### DETAILED DESCRIPTION OF PREFERRED EMBODIMENTS

Materials and Methods

Experimental Design

Male albino mice were used throughout the study, housed in a environmentally regulated room on a dark-light cycle of 12 hours with free access to food and water. Principles of laboratory animal care (NIH publication no. 85-23, revised 1985) were followed, as well as specific Spanish regulations. Animals were made diabetic with a single subcutaneous injection of 200 mg alloxan/kg body weight (66 mg/ml) in 0.1 M citrate buffer, pH 4.5, at day 0 of the experiment. Control mice were injected with the same volume of vehicle also at day 0 of the experiment.

Mice were identified as diabetic on the basis of blood glucose levels (higher than 16 mM at least 4 days after alloxan treatment). Animals were divided into subgroups as required by the experiment (control, control+lutein, diabetic, diabetic+lutein, diabetic+insulin, diabetic+insulin+lutein). Lutein was administered daily at a dose of 0.2 mg/kg p.o since day 4 after alloxan or citrate buffer injection until the end of the experiment. Insulin treated animals were injected daily with 500 mU/g body weight since day 4 until the end of the experiment. Mice were killed by cervical dislocation on day 14, and hippocampus and retina were removed and homogenized in pre-chilled 0.2 M potassium phosphate buffer, pH 7.0. These homogenates were used to assay protein, GPx activity and MDA and GSH concentrations. Samples were kept frozen (−80° C.) until biochemical assays were performed. Blood was taken from the tail vein daily to assay blood glucose levels. Glycated hemoglobin was determined in blood samples obtained by heart puncture immediately before killing the animals.

Biochemical Assays

MDA, a lipid peroxidation product, concentration was measured by liquid chromatography according to a modification of the method of Richard et al. (1992) as previously described (Romero et al., 1998). Briefly, 0.1 ml of sample (or standard solutions prepared daily from 1,1,3,3-tetramethoxypropane) and 0.75 ml of working solution (thiobarbituric acid 0.37% and perchloric acid 6.4%; 2/1, v/v) were mixed and heated to 95° C. for 1 h. After cooling (10 min in ice water bath), the flocculent precipitate was removed by centrifugation at 3,200×g for 10 min. The supernatant was neutralized and filtered (0.22 μm) prior to injection on an ODS column 5 μm column (250×4.6 mm). Mobile phase consisted in 50 mM phosphate buffer (pH 6.0): methanol (58:42, v/v). Isocratic separation was performed with 1.0 ml/min flow (HPLC System 325, Kontron) and detection at 532 nm (UV/VIS HPLC Detector 332, Kontron). Calibration curves were run daily.

GPx activity was assayed as reported by Lawrence et al. (1978) towards hydrogen peroxide. The disappearance of NADPH was followed spectrophotometrically at 340 nm. The reaction mixture consisted of 240 mU/ml of glutathione disulfide reductase, 1 mM GSH, 0.15 mM NADPH in 0.1 M potassium phosphate buffer, pH 7.0, containing 1 mM EDTA and 1 mM sodium azide; 50 μl sample were added to this mixture and allowed to equilibrate at 37° C. for 3 min. reaction was started by the addition of 1.5 mM hydrogen peroxide to adjust the final volume of the assay mixture to 1 ml.

The GSH content of eye homogenate was quatitated by the method of Reed et al. (Reed et al., 1980). Briefly, eyes were homogenized in pre-chilled medium containing phosphate buffer (pH 7.0) and perchloric acid (PCA). Suspensions were centrifuged at 14,000×g and the resulting supernatants were collected and stored at ×80° C. The samples were mixed with a solution of iodoacetic acid and Sanger reagent (1-fluor-2,4-dinitrobencene). These products are quickly separated by HPLC which allows the quantification of nanomolars levels of GSH.

Protein content was measured by means of the Lowry method (Lowry et al., 1951).

Morris Water Maze Training

The experiment animals were trained in a Morris water maze during the last 5 days of the experiment to evaluate spatial learning and memory (in this case animals were kept alive for three weeks). The water maze consisted of a tank of water, 1.2 meters in diameter, divided clockwise into four quadrants A, B, C and D. The tank was filled with clear water at room temperature. One centimeter below the surface of the water, a rigid platform, 11 cm in diameter, was fixed in Quadrant A. The apparatus was kept in the same position in a room and the experimenter was situated in the same location for all trials. Mice were carefully dropped into the maze from a predetermined location near the wall of the water tank.

Mice were trained for 5 days on negotiating spatial bias, receiving 3 trials each day. We measured the duration from the time the mouse was dropped into the maze to the time the mouse climbed onto the platform (latency time). The latency was recorded manually using a stopwatch. The maximum time allowed for the mouse to find the platform was 3 minutes. If they could not reach the platform within 3 minutes, they were led onto the platform. The percentage of time spent in each of the pool quadrants was also recorded.

NF-Kappa B

NF-κB activity was determined with TransAm Kit (Active Motif) that combines an ELISA test with an specific assay for transcription factors. Briefly, it consists in adding the sample to wells that contain the consensus sequence of NF-κB. Primary antibody recognize an epitope that is only accessible when NF-κB is binded to DNA. The sencondary antibody produces a colorimetic reaction that can be followed spectrophotometrically.

Immunostaining

All animals were killed with a barbiturate overdose; each group had n>8. After intracardial perfusion with 4% paraformaldehyde, brains were cut into 40-μm-thick sections (200 μm, 1 every sixth section, used for electron microscopy analysis) with a vibratome (Leica VT100M).

Sections were immersed free-floating in 0.1 M phosphate buffer plus 0.05 % sodium azide. Twenty-four-well plates were used to keep the sections separate and preserve the order of the series. Sections that were 40 μm thick were used for cell counting. Incubations were performed in 0.1 M phosphate buffer with 0.2% Triton X-100 and 10% normal goat serum. Sections were blocked by incubating for 30 min in a solution of 3% hydrogen peroxide for diaminobenzidine (Sigma) staining. BrdU immunohistochemistry was preceded with DNA denaturation of 25 min of incubation in 2 M HCl at 37° C. The primary antibodies used were 1:200 mouse anti-BrdU (Dako).

Single-label BrdU immunohistochemistry was performed for stereology counts, and the secondary antibody was a 1:300 biotinylated horse anti-mouse IgG (Vector Laboratories) followed by the peroxidase-based ABC system (Pierce) using diaminobenzidine as the chromogen. For double immunohistochemistry, we used 1:200 anti-rabbit Cy² and 1:1,000 anti-mouse Cy³ (Jackson ImmunoResearch).

Cell Counting. BrdU-positive cells were counted in sections spaced 400 μm apart by using a Nikon Eclipse E200 with a Nikon 40X Plan objective (0.65 numerical aperture) throughout the granule cell layer. At least 12 sections in the hippocampi and 4 in the OBs were studied in each animal. The same areas and number of sections were studied for all of the animals and all of the experimental groups. We considered as BrdU⁺ those nuclei completely filled with diaminobenzidine product or fluorescent marker or showing patches of variable intensity. We used the optical dissector technique to estimate the cell density of BrdU⁺ cells in the granule cell layer. The granule cell area was traced by using a camera lucida (Olympus), and drawings were scanned with an Hewlett-Packard ScanJet 5300C and processed with Scion (Frederick, Md.) IMAGE BETA 4.0.2 software to determine the area of the granule cell layer at each level examined. Data were expressed as total, for the whole structure, and also subanalyzed in three regions: anterior, middle, and posterior.

Statistical Analysis

The results are presented as mean values±standard deviation. Statistical significances were assessed by ANOVA followed by the t-Student test. The level of significance was set at p<0,05. The water maze data were analyzed using ANOVA with repeated measures.

Results

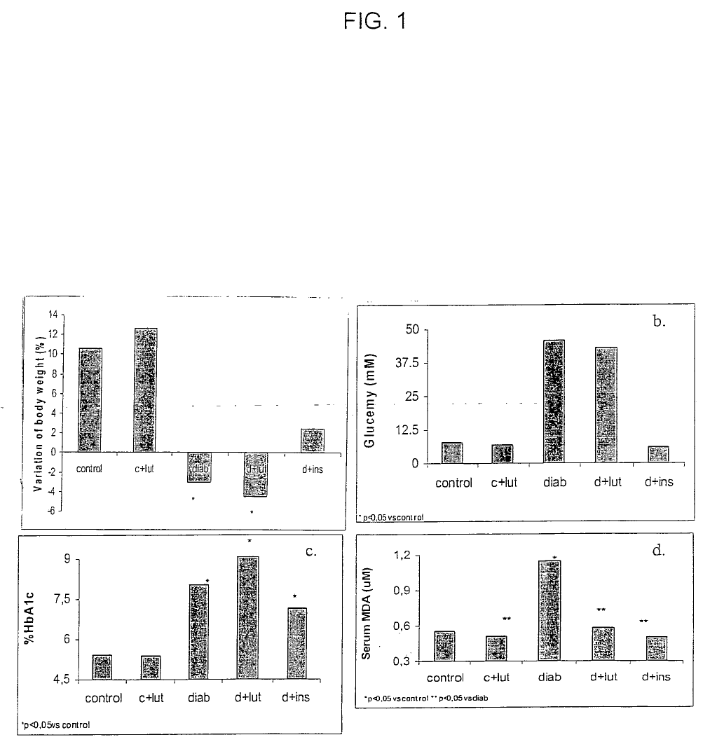

The diabetes model in mice, fourteen days after alloxan injection was used to achieve hyperglycemia, confirmed by: body weight changes (FIG. 1a), blood levels of glucose (FIG. 1b) and glycated hemoglobin levels (FIG. 1c. Lutein treatment did not alter the hyperglycemic status of alloxan diabetic mice. Malondialdehyde concentration in blood, was elevated in diabetic animals, confirming the overall existence of an oxidative burden (FIG. 1d).

Retina

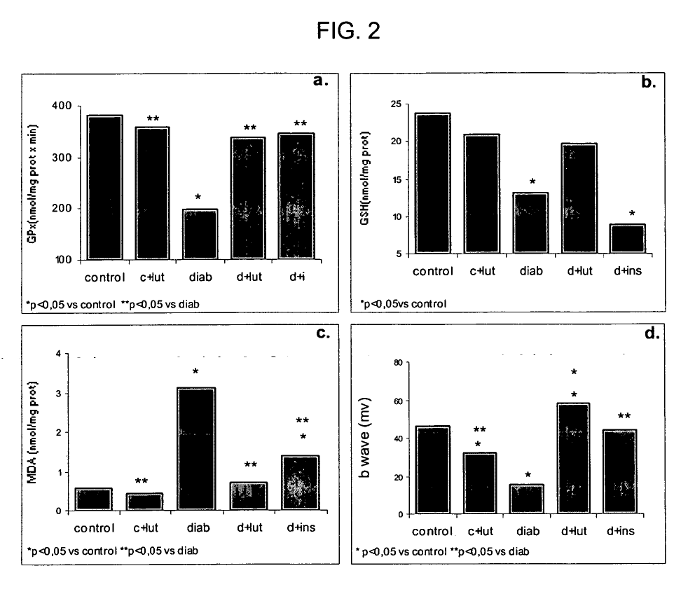

The biochemical and functional changes in the retina of diabetic mice, and the ability of Lutein, a natural antioxidant, to reverse these effects have been studied, compared to the effect of insulin therapy. GPx activity (FIG. 2a), the key enzymatic activity metabolizing cytosolic and mitochondrial hydrogen peroxide, and GSH concentration (FIG. 2b) were assayed in eye homogenate without lens and both decreased in the diabetic condition, whereas ocular MDA concentration (FIG. 2c) was higher than controls. Maximal electroretinogram (ERG) amplitude (mostly b-wave) decreased in diabetic animals respect to controls. Lutein administration (100 mg/kg p.o.) or insulin treatment (500 mU/g body weight s.c.) on days 4-13, restored MDA levels in serum and ocular tissue, the former having no effect on glycemia. Lutein protected retinal GPx activity and restored ERG amplitude (FIG. 2d) in diabetic animals to control values, whereas insulin partially reversed the effects of diabetes on GPx and ERG.

Hippocampus

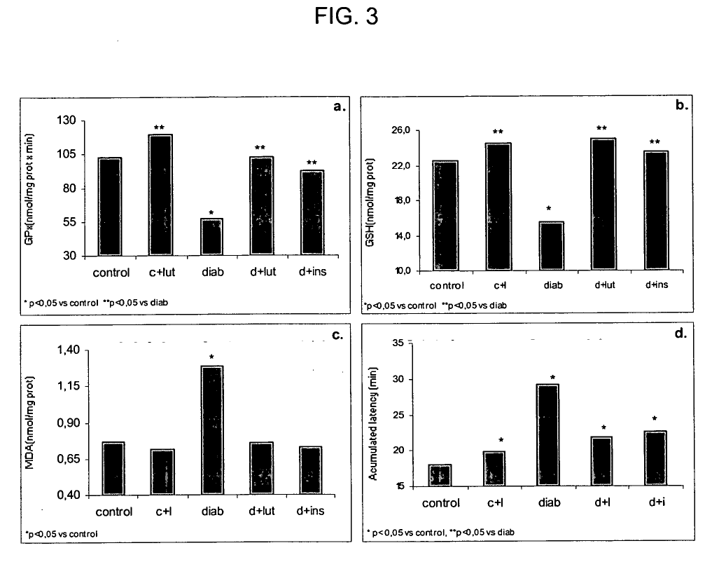

GPx activity (FIG. 3a), and GSH content (FIG. 3b) were assayed in hippocampus homogenate, and both decreased in the diabetic condition, whereas hippocampal MDA concentrations (FIG. 3c) were higher than controls. Lutein administration (0.2 mg/kg p.o.) or insulin treatment (500 mU/g body weight s.c.) on days 4-13 restored MDA levels in hippocampus, the former having no effect on glycemia. Lutein and insulin restored GPx activity and GSH content to control values.

When examined for spatial learning ability, mice in the control groups eventually learned the spatial location of the platform after 3 trials, as measured by the progressive decrease in the time taken to climb onto the platform (latency). The diabetic mice were significantly retarded in the acquisition of the task compared to control subjects (FIG. 3d). The control mice spent the majority of their time swimming in the quadrant where the platform had been located, while the stimulated mice distributed their time more uniformly in all four pool quadrants.

Impaired hippocampal-dependent learning (water maze) was restored to control values with both treatments.

NF-κB

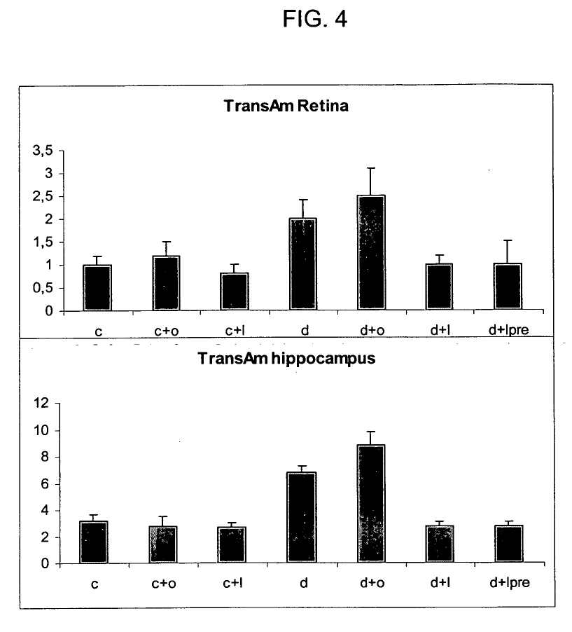

Nuclear factor κB expression is increased in the retina (FIG. 4a) and hippocampus (FIG. 4b) of diabetic animals, when compared with untreated controls and lutein-treated mice. Lutein treatment before and after diabetes induction, decreased NF-κB expression both in retina and hippocampus.

Neurogenesis

We decided to study neurogenesis in hippocampi from diabetic mice because it is known that the alteration of hippocampal NF-κB as the result of ROS does have significant implications in neurogenesis. A recent study (Saravia et al. 2004) demonstrated a strong reduction in cell proliferation rate in the subgranular zone of the dentate gyrus and the subventricular zone of diabetic mice killed 20 days after streptozotocin administration. 17beta-oestradiol restored brain cell proliferation in the diabetic mouse brain. Previous studies from our lab (Herrera et al., 2003) demonstrated that neurogenesis is impaired in a model of chronic alcoholism in rats and that ebselen, an antioxidant that shares with lutein the property of being a peroxynitrite scavenger, was able to prevent this alteration. We have also demonstrated the benefitial effects of ebselen in the retina of diabetic mice. In this study, ebselen and lutein restored glutathione peroxidase activity of the retinas and b-wave amplitude of the electroretinogram of diabetic animals to control values (Miranda et al., 2004).

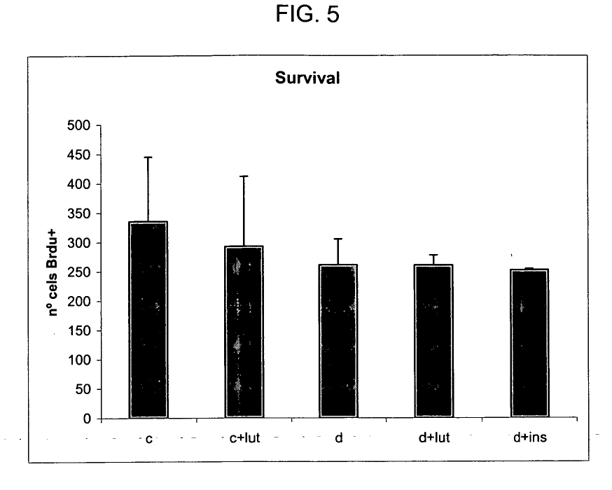

Current studies in our lab confirm that the survival of new neurons is not affected in this model of experimental diabetes (FIG. 5), as seen in the literature, but the proliferation of hippocampal stem cells was affected. Preliminary data seem to indicate that lutein is able to prevent the impairment in proliferation. In fact, the impaired neurogenesis is related to cognitive deficits and in this model of diabetes we have observed that the administration of lutein diminished the escape latencies of diabetic mice in the Morris water maze, as previously described (cf. FIG. 3d).

Discussion

Lutein is one of the two major carotenoids in the human macula and retina (Handelman, 1988). These pigments are found in various coloured fruits and green leafy vegetables. Some epidemiological evidence suggest a beneficial role for lutein in the prevention of age-related macular degeneration (The Eye-Disease Case Control Study Group, 1993). Diabetic retinopathy is the first cause of adult blindness in developed countries, and though strict glycemic control is desirable to prevent complications, this is not always achievable. Thus, adjuvant therapies are needed to help in preventing or delaying the onset of diabetic complications. The biochemical and functional changes in the retina of diabetic mice, and the ability of lutein, a natural antioxidant, to reverse these effects have been studied, compared to the effect of insulin therapy. Recent studies (Hagino et al., 2004) confirm vascular change of the retinal capillaries becomes also a prominent feature during the processes of aging and it has a positive correlation with the vascular change of hippocampal capillary. In the present study we also investigated the alterations in terms of oxidative stress of hippocampus from diabetic mice and for the first time we have studied the effect of Lutein on diabetic hippocampus.

Because of high levels of polyunsaturated lipids, nervous tissue is markedly sensitive to oxygen free radical damage. Our results show that after 2 weeks of diabetes, MDA levels in retina and hippocampus diabetic mice are increased when compared to in controls, thus confirming the role of lipid peroxidation in diabetes, and that lutein and insulin are able to prevent this effect. Also, it is known that MDA determination by high-pressure liquid chromatography is a good marker of oxidative stress implication in a pathological process (Halliwell et al 2000). Glutathione peroxidase activity and glutathione content were decreased after 2 weeks of diabetic condition, not only in retina but also hippocampus.

Apoptosis occurs in diabetes as well as in its chronic complications, like retinopathy (Barber et al 1998) and more recently has been demonstrated apoptosis in neuropathy (Cellek et al 2004) and in the hippocampus of type 1 diabetic rats (Li et al 2002). Peroxynitrite (ONOO(−)), the product of the reaction between nitric oxid (NO) and superoxide has been suggested to be involved in apoptotic cell death, therefore, cells that constitutively express NO synthase, such as neurons, may be more vulnerable to ONOO(−)-induced cell death in conditions favoring the production of superoxides (Cowell et al 2004).

Therefore, lutein, and antioxidant with properties as a scavenger of peroxynitrites (Panasenko et al 2000) seems to be an appropriate treatment for the impairments observed in this study.

Diabetes mellitus correlates with several brain disturbances, including hypersensitivity to stress, cognitive impairment, increased risk of stroke and dementia. Within the central nervous system, the hippocampus is considered a special target for alterations associated with diabetes.

Neurogenesis is a plastic event restricted to few adult brain areas: the subgranular zone of the dentate gyrus and the subventricular zone. The majority of these cells eventually differentiate into granule neurons. Alterations in neurogenesis within the adult hippocampus have been shown to occur through a change in either the number of proliferating cells or the survival of newly generated cells. Thus, the observed decrease in proliferating cells within the dentate gyrus of the diabetic animals suggests that a reduction in newly formed granule neurons may occur. Neurogenesis has been implicated in hippocampal-dependent learning. In general, factors that reduce neurogenesis (glucocorticoids, stress, aging) decrease learning, whereas factors that enhance neurogenesis (estrogen, enriched environment) increase learning (Gould et al., 1999).

A recent study (Saravia et al. 2004) demonstrated a strong reduction in cell proliferation rate in the subgranular zone of the dentate gyrus and the subventricular zone of diabetic mice killed 20 days after streptozotocin administration. 17Beta-oestradiol restored brain cell proliferation in the diabetic mouse brain. We have demonstrated that proliferation in hippocampus is decreased only two weeks after the induction of diabetes and again lutein is able to restore this alteration.

Latency to escape to a hidden platform in the Morris Water Maze is used widely to test spatial memory, a hippocampus-dependent task. Lupien et al. (2003) demonstrated that IGF-I can act across the B-CNS-B to prevent loss of cognition-related performance in the water maze independently of ongoing hyperglycemia and reduction in brain and whole body weight in diabetic rats. Other studies suggest that cognitive impairment in type 1 diabetes is associated with a duration-related apoptosis-induced neuronal loss (Li et al. 2002). They examined hippocampal abnormalities in the spontaneously type 1 diabetic BB/W rat. The Morris water maze test showed significantly prolonged latencies in 8-month diabetic rats not present at 2 months of diabetes. These abnormalities were associated with DNA fragmentation, positive TUNEL staining, elevated Bax/Bcl-x(L) ratio, increased caspase 3 activities and decreased neuronal densities in diabetic hippocampi. Our results are in agreement with previous studies that have described consistently displayed performance deficits in Morris water maze of diabetic rodents (Biessels et al 1996), and that insulin treatment from the onset of diabetes can prevent this learning impairment (Biessels et al 1998), but we show for the first time, that cognitive impairments start only after 3 weeks of diabetes induction, and that the administration of a natural antioxidant without hypoglycemiant properties, is also able to prevent this deficit.

Although a proper glycemic control is desirable to reduce the development of diabetic complications, it is not sufficient to prevent them completely, the results herein, allow us to propose lutein as a coadyuvant treatment of central nervous system complications in diabetes.

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art who have the disclosure before them will be able to make modifications and variations therein without departing from the scope of the invention.

### REFERENCES

Aragno M, Mastrocola R, Brignardello E, Catalano M, Robino G, Manti R, Parola M, Danni O, Boccuzzi G. Dehydroepiandrosterone modulates nuclear factor-kappaB activation in hippocampus of diabetic rats. Endocrinology. 2002 Sep; 143(9):3250-8.

Barber A J.; Lieth, E.; Khin, S A.; Antonetti, D A.; Buchanan, A G.; Gardner T W.; and The Penn State Research Group. (1998). Neural apoptosis in the retina during experimental and human diabetes. J. Clin. Invest.; Vol. 102, N^{o} 4, August, 783-791.

Baydas G, Reiter R J, Yasar A, Tuzcu M, Akdemir I, Nedzvetskii V S. Melatonin reduces glial reactivity in the hippocampus, cortex, and cerebellum of streptozotocin-induced diabetic rats.: Free Radic Biol Med. 2003 Oct 1;35(7):797-804.

Baynes J W, Thorpe S R. The role of oxidative stress in diabetic complications. Current opinion in Endocrinology 1996; 3:277-284.

Baynes, J W.; Thorpe, S R. Role of oxidative stress in Diabetic Complications. A New Perspective on an Old Paradigm. Diabetes 1999; 48: 1-9.

Biessels G J, Kamal A, Urban I J, Spruijt B M, Erkelens D W, Gispen W H. Water maze learning and hippocampal synaptic plasticity in streptozotocin-diabetic rats: effects of insulin treatment. Brain Res. 1998 Jul 27;800(1):125-35.

Cellek S, Qu W, Schmidt A M, Moncada S. Synergistic action of advanced glycation end products and endogenous nitric oxide leads to neuronal apoptosis in vitro: a new insight into selective nitrergic neuropathy in diabetes. Diabetologia 2004 Feb;47(2):331-9.

Coleman E, Judd R, Hoe L, Dennis J, Posner P. 2004) Effects of diabetes mellitus on astrocyte GFAP and glutamate transporters in the CNS. Glia. Nov 1;48(2):166-78.

Cowell R M, Russell J W. Nitrosative injury and antioxidant therapy in the management of diabetic neuropathy. J Investig Med. 2004 Jan;52(1):33-44.

Gage, F. H. Mammalian neural stem cells. Science 2000, 287, 1433-1438.

E. Gould, P. Tanapat, N. B. Hastings and T. J. Shors, Neurogenesis in adulthood: a possible role in learning. Trends Cog. Sci. 3 1999, pp. 186-192.

Grossman H. Does diabetes protect or provoke Alzheimer's disease? Insights into the pathobiology and future treatment of Alzheimer's disease. CNS Spectr. 2003 Nov;8(11):815-23.

Hagino N, Kobayashi S, Tsutsumi T, Horiuchi S, Nagai R, Setalo G, Dettrich E. Vascular change of hippocampal capillary is associated with vascular change of retinal capillary in aging. Brain Res Bull. 2004 Feb 15;62(6):537-47.

Halliwell, B. (2000). Oxidative stress markers in human disease: application to diabetes and to evaluation of the effects of antioxidants. In: Antioxidants in Diabetes Management. Packer, L., Rosen, P., Tritschler, H. J., King, G. L. and Azzi A., Eds., New York: Marcel Dekker, 2000, pp. 33-52.

Handelman, G J, Dratz, E A, Reay, C C, Van Kuijk, J G. Carotenoids in the human macula and whole retina. Invest Ophthalmol Vis Sci 1988; 29:850-5.

Herrera D G, Yague A G, Johnsen-Soriano S, Bosch-Morell F, Collado-Morente L, Muriach M, Romero F J, Garcia-Verdugo J M. Selective impairment of hippocampal neurogenesis by chronic alcoholism: protective effects of an antioxidant. Proc Natl Acad Sci USA. 2003, 100(13):7919-24.

Jackson-Guilford J, Leander J D, Nisenbaum L K. The effect of streptozotocin-induced diabetes on cell proliferation in the rat dentate gyrus. Neurosci Lett. 2000 Oct 27;293(2):91-4.

Jarvis M J. Does caffeine enhance absolute levels of cognitive performance? Psychopharmacology (Berl) 1993; 110(1-2):45-52.

Joseph J A, Shukitt-Hale B, Denisova N A, et al. Long-term dietary strawberry, spinach, or vitamin E supplementation retards the onset of age-related neuronal signal-transduction and cognitive behavioral deficits. J Neurosci 1998;18(19):8047-55.

Kowluru R A, Koppolu P, Chakrabarti S, Chen S. Diabetes-induced activation of nuclear transcriptional factor in the retina, and its inhibition by antioxidants. Free Radic Res. 2003 Nov;37(11):1169-80.

Lawrence, R. A., Parkhill, L. K., Burk, R. F. (1978). Hepatic cytosolic non-selenium dependent glutathione peroxidase activity: its nature and the effect of selenium deficiency. J. Nutr. 108, 981-87.

Li Z G, Zhang W, Grunberger G, Sima A A. Hippocampal neuronal apoptosis in type 1 diabetes. Brain Res. 2002 Aug 16;946(2):221-31.

Li Z G, Sima A A. C-peptide and central nervous system complications in diabetes. Exp Diabesity Res. 2004 Jan-Mar;5(1):79-90.

Lowry, O. H., Rosebrough, N. J., Farr, A. L. and Randall, R. J. (1951). Protein measurement with the Folin phenol reagent. J. Biol. Chem. 193, 265-75.

Lupien S B, Bluhm E J, Ishii D N. Systemic insulin-like growth factor-I administration prevents cognitive impairment in diabetic rats, and brain IGF regulates learning/memory in normal adult rats.: J Neurosci Res. 2003 Nov 15;74(4):512-23.

Miranda M, Muriach M, Johnsen S, Bosch-Morell F, Araiz J, Roma J, Romero F J. Oxidative stress in a model for experimental diabetic retinopathy: treatment with antioxidants. Arch Soc Esp Oftalmol. 2004 Jun;79(6):289-94.

Panasenko O M, Sharov V S, Briviba K, Sies H. Interaction of peroxynitrite with carotenoids in human low density lipoproteins. Arch Biochem Biophys. 2000 Jan. 1;373(1):302-5.

Perrig W J, Perrig P, Stahelin H B. The relation between antioxidants and memory performance in the old and very old. J Am Geriatr Soc 1997;45(6):718-24.

Reed, D. J., Babson, J. R., Beatty, P. W., Brodie, A. E., Ellis, W. W. and Potter, D. W. (1980). High-performance liquid chromatography analysis of nanomole levels of glutathione, glutathione disulfide, and related thiols and disulfides. Anal. Biochem. 106(1), 55-62.

Richard, M. J., Guiraud, P., Meo, J. and Favier, A. (1992). High performance liquid chromatography separation of malondialdehyde thiobarbituric acid adduct in biological materials (plasma and human cell) using a commercially available reagent. J. Chromatogr. 20 577, 9-18.

Romero, M. J., Bosch-Morell, F., Romero, B., Rodrigo, J. M., Serra, M. A. and Romero, F. J. (1998). Serum malondialdehyde: possible use for the clinical management of chronic hepatitis C patients. Free Radical Biol Med 25: 993-997.

Saravia F, Revsin Y, Lux-Lantos V, Beauquis J, Homo-Delarche F, De Nicola A F. Oestradiol restores cell proliferation in dentate gyrus and subventricular zone of streptozotocin-diabetic mice. J Neuroendocrinol. 2004 Aug;16(8):704-10.

Shors, T. J., Miesegaes, G., Beylin, A., Zhao, M., Rydel, T. & Gould, E. (2001) Nature 410, 372-376.

Solfrizzi V, Panza F, Torres F, et al. High monounsaturated fatty acids intake protects against age-related cognitive decline. Neurology 1999;52(8):1563-9

The Eye-Disease Case Control Study Group. Antioxidant status and neovascular age-related macular degeneration. Arch Ophthalmol 1993; 111: 104-109.

We claim:

1. A method of ameliorating cognitive decline in mammals, comprising administering a therapeutic amount of a carotenoid.

2. A method as defined in claim 1, wherein the carotenoid comprises lutein.

3. A method as defined in claim 1, wherein the mammal is human.

4. A method of ameliorating oxidative stress in mammals, comprising administering a therapeutic amount of a carotenoid.

5. A method as defined in claim 4, wherein the carotenoid comprises lutein.

6. A method as defined in claim 4, wherein the mammal is human.

7. A method of reducing the levels of glutathione and glutathione peroxydase activity in the hippocampus of mammals, comprising administering a therapeutic amount of a carotenoid.

8. A method as defined in claim 7, wherein the carotenoid comprises lutein.

9. A method as defined in claim 7, wherein the mammal is human.

10. A method of increasing neurogenesis in mammals, comprising administering a therapeutic amount of a carotenoid.

11. A method as defined in claim 10, wherein the carotenoid comprises lutein.

12. A method as defined in claim 10, wherein the mammal is human.
